(19) United States
(12) Plant Patent Application Publication
(10) Pub. No.: US 2015/0313050 P1
(43) Pub. Date: Oct. 29, 2015

(54) Grapevine 'IFG Twenty'

(50) ;

(71) Applicant: David Cain, Bakersfield, CA (US)

(72) Inventor: David Cain, Bakersfield, CA (US)

(21) Appl. No.: 14/120,084

(22) Filed: Apr. 25, 2014

(51) Int. Cl. A01H 5/00

(52) U.S. Cl. PLT/206

(57) ABSTRACT

This invention is a new and distinct grapevine variety denominated ‘IFG Twenty’. The new grapevine is characterized by producing naturally large seedless blue-black berries having a narrow ellipsoidal to cylindrical shape with a strong fruity labrusca flavor. Fruits normally ripen mid to late season about early to mid-September near Delano Calif. Fruits are fairly low in acidity, with medium dense firm texture, occasionally noticeable seed trace. Vines are productive and can be spur pruned.

### LATIN NAME OF THE GENUS AND SPECIES CLAIMED

Vitis interspecific hybrid

### VARIETY DENOMINATION

‘IFG Twenty’

### BACKGROUND OF THE INVENTION

The new and distinct grapevine described and claimed herein originated from a hand pollinated cross of the IFG 01032-067-222, an unnamed seedless selection from the IFG breeding program, and the A2798 (unnamed interspecific selection from the University of Arkansas) hybridized in May 2006. The abortive seed traces were subsequently embryo cultured and the resulting population of plants was planted in the field in April 2007. The present variety of grapevine was selected as a single plant in September 2008 and was first asexually propagated by hardwood cuttings in December 2008 near Delano, Kern County, Calif. The resulting propagules were planted during April 2009 near Delano, Kern County, Calif. and were found to reproduce true-to-type through at least two generations of asexual reproduction.

### BRIEF SUMMARY OF THE INVENTION

The new grapevine ‘IFG Twenty’ is characterized by producing naturally large seedless blue-black berries having a narrow ellipsoidal to cylindrical shape with a strong fruity labrusca flavor. Fruits normally ripen mid to late season about early to mid-September near Delano Calif. Fruits are fairly low in acidity, with medium dense, firm texture, with occasionally noticeable seed traces. Attachment of the berry to the pedicel is somewhat weak. The variety stores and ships well and is suitable for long-term storage and long distance shipping. Vines are vigorous, very productive and can be pruned to short spurs.

The new grapevine differs from its maternal parent the IFG 01032-067-222 by having blue-black rather than light red berries and by having a very strong fruity labrusca type flavor as opposed to the neutral flavor of the IFG 01032-067-222. The berries of ‘IFG Twenty’ are more elongated than the IFG 01032-067-222 and the peduncle is less lignified. ‘IFG Twenty’ can be distinguished from its pollen parent, A2798, by having a more elongated and less firm berry, by having larger berry and cluster size and by having much better resistance to tip cracking of berries when fully ripe as compared to the A2798.

Grape breeders have used several native American grape species to improve hardiness, disease and insect resistance as well as incorporate aromatic flavors into the vinifera species. Previously introduced interspecific varieties have had very limited commercial success due to small fruit size, large seed traces, slip-skin texture or lack of firmness. The new grapevine variety is being introduced because of its unique combination of late ripening, labrusca flavor combined with firm texture, seedlessness, good adhesion of skin and flesh and naturally large berry size.

### BRIEF DESCRIPTION OF THE FIGURE

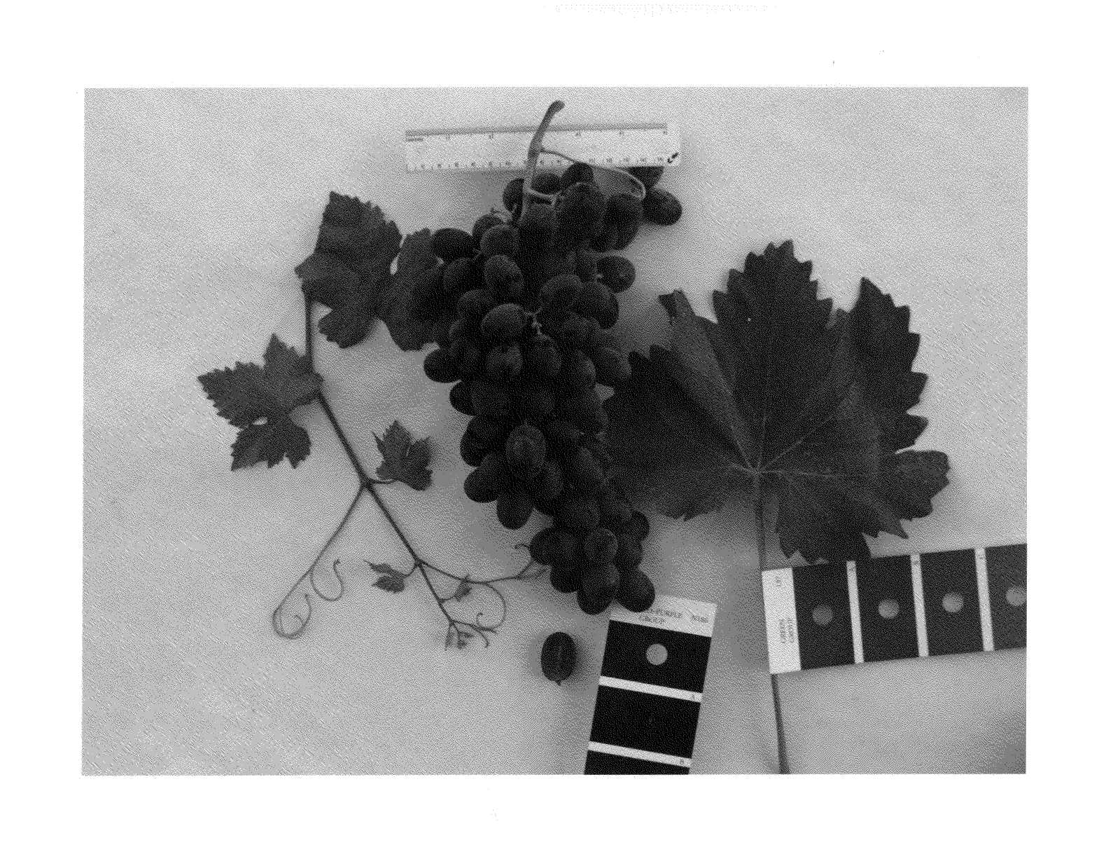

The accompanying photographic illustration in FIG. 1 illustrates in full color ‘IFG Twenty’. The photograph was taken outdoors with indirect lighting. The colors are as nearly true as is reasonably possible in a color representation of this type.

### DETAILED BOTANICAL DESCRIPTION OF THE INVENTION

Throughout this specification, color names beginning with a small letter signify that the name of that color, as used in common speech, is aptly descriptive. Color names beginning with a capital letter designate values based upon R.H.S. Colour Chart, published by The Royal Horticultural Society, London, England.

Throughout this specification subjective description values conform to those set forth by the International Plant Genetic Resources Institute publication ‘Descriptors for Grape’ (vitis spp.) (1983) which was developed in collaboration with the Office International de la Vigne et du Vin (OIV) and the International Union for the Protection of New Varieties of Plants (UPOV).

The descriptive matter which follows pertains to ‘IFG Twenty’ plants grown in the vicinity of Delano, Kern County, Calif. during 2012 and is believed to apply to plants of the variety grown under similar conditions of soil and climate elsewhere:
Vine:
   General.—Size — Large Vigor — Vigorous Density of foliage — Dense Productivity — Very productive Root stock — Own root Training method — Typically spur pruned leaving 2 bud spurs.
   Trunk.—Trunk diameter of 4-year-old vines at 30 cm above the soil line — About 5.7 cm Shape — Stocky to medium Straps — Short — split Surface texture — Medium Inner bark color — Greyed orange: can be any of the following colors; 177A and B.
Shoots:
   Young shoot.—Form of tip — Wide open Distribution of anthocyanin coloration of tip — Absent Intensity of anthocyanin coloration of tip — Absent Density of prostrate hairs of tip — Medium to Dense Density of erect hairs of tip — Absent Color — Yellow — Green: can be any of the following colors; 144A, and 146B and C.
   Woody shoot (mature canes).—Shape — Medium Internode length — Medium; About 7.6 cm Width at node — About 2.0 cm Cross section — Elliptic Surface — Striate Main color — Greyed orange: can be any of the following colors; 166A and B and C and D Density of erect hairs on nodes — None Density of erect hairs on internodes — None Growth of axillary shoots — Medium long; approximately; 19.2 cm.
   Flowering shoot.—Vigor during flowering — Strong Attitude during flowering on shoots not tied — Semi-drooping to Drooping Color — dorsal side of internodes — Green with Red stripes Color — ventral side of internodes — Green Color — dorsal side of nodes — Green Color — ventral side of nodes — Green Density of prostrate hairs of nodes — Very sparse Density of erect hairs of nodes — Very sparse Density of prostrate hairs on internode — None Density of erect hairs on internode — None Anthocyanin coloration of buds — Absent.
   Tendrils.—Distribution on the shoot (at full flowering) — Discontinuous Length of Tendril — Medium; About 16.9 cm Thickness — Medium Color — Yellow-Green; can be any of the following colors; N144A and C, and 146B and C Form — Trifurcated Number of consecutive tendrils — 2.
Leaves:
   Young leaves.—Color of upper surface of first four distal unfolded leaves — Green Average intensity of anthocyanin coloration of six distal leaves prior to flowering — Absent Density of prostrate hairs between veins (lower surface) — Very sparse Density of prostrate hairs on veins (lower surface) — Very sparse Density of erect hairs between veins (lower surface) — Absent Density of erect hairs on veins (lower surface) — Sparse.
   Mature leaves.—Average length — About 14.5 cm Average width — About 18.0 cm Mature leaf size — Medium large Shape of blade — Wedge-shaped Number of lobes — 5 Anthocyanin coloration of main veins on upper side of blade — Absent to Very weak Mature leaf profile — Undulate Blistering surface of blade upper surface — Very weak Leaf blade tip — In the plane of the leaf Undulation of margin — Medium Thickness — Medium Undulation of blade between main and lateral veins — Overall Shape of teeth — Mixture of both sides straight and both sides convex Length of teeth — Medium Ratio length/width of teeth — Medium Shape of upper lateral sinuses — Closed to Lobes slightly overlapping Depth of upper lateral sinuses — Shallow General shape petiole sinus — Half open to Slightly open Shape of base of upper leaf sinuses — U-shaped Tooth at petiole sinus — Absent Density of prostrate hairs between veins on lower surface of blade — Absent to Very sparse Density of erect hairs between veins on lower surface of blade — Absent Density of prostrate hairs on main veins on lower surface of blade — Sparse Density of erect hairs on main veins on lower surface of blade — Sparse to Medium Density of prostrate hairs on main veins on upper surface of blade — Very sparse Density of erect hairs on main veins on upper surface of blade — None Autumn coloration of leaves — Leaves can be a single color or combination of colors, in a mottled pattern or on the edges of the leaves; Yellow; 13A and B, and Greyed orange; 175A, and Greyed purple; 183A and B.
   Upper surface.—Color — Can be any of the following colors; 137A and B Anthocyanin coloration of main veins — Absent Surface appearance — Dull Blistering surface of blade — Very weak.
   Lower surface.—Color — Can be any of the following colors; 138A and B Anthocyanin coloration of main veins (lower surface) — Absent Glossiness — Weak Surface texture — Smooth Surface appearance — Dull.
   Petiole.—Length — About 12.5 cm Length of petiole compared to middle vein — Slightly shorter Density of prostrate hairs on petiole — None Density of erect hairs on petiole — None.
   Buds.—Bud fruitfulness — Basal: Mostly fruitful Position of first fruitful shoot on previous season cane — 1^{st} to 3^{rd} node Time of bud burst — Midseason; about Mar. 15, 2012.
Flowers:
   General.—Flower sex — Hermaphrodite Length of first inflorescence — Medium to Long; About 25.5 cm long by 13.3 cm wide Position of first flowering and fruiting node — 4^{th} to 5^{th} node (current season growth) Number of inflorescence per flowering shoot — 1.1 to 2 Time of bloom — Late as compared with similar varieties in the growing area of Delano, Calif. Date of full bloom — About Mar. 18, 2012.
Fruit:
   General.—Ripening period — Mid to late season; approximately Sep. 18, 2012 Use — Fresh market Keeping quality — Excellent Resistance to — Insects: Average typical of Vitis vinifera species. Diseases: Average typical of Vitis vinifera species. Refractometer test — soluble solids: About 18.6 Brix Brix/acid — About 38.0 Titratable acidity — About 0.49 Juice pH — About 3.64.
   Cluster.—Mature cluster length (peduncle excluded) — About 35.6 cm Mature cluster width — About 17.8 cm Mature cluster weight — About 1236 g Bunch density — Loose to Medium loose Number of berries — About 208 Form — Conical.
   Peduncle.—Lignification of peduncle — Weak Length of peduncle — Medium long. Approximately 2.2 cm.
   Berry.—Uniformity of size — Uniform Single berry weight — About 6.6 g natural; to about 7.9 g when treated with gibberellic acid Shape — Narrow ellipsoidal to cylindrical Seeds — Seed traces occasionally noticeable Cross section — Circular Berry dimensions — Longitudinal axis: About 2.7 cm. horizontal axis: About 1.9 cm Berry firmness — Medium firm Particular flavor — Labrusca Bloom (cuticular wax) — Medium to heavy Berry separation from pedicel — Medium to Easy Skin color (without bloom) — Blue-black: can be any of the following colors; N186 B and C.
   Skin.—Thickness — Medium Texture. — Medium tough Reticulation — Absent Tenacity — Tenacious to flesh.

What is claimed:

1. A new and distinct variety of grapevine as herein illustrated and described.
